(19) United States
(12) Patent Application Publication
(10) Pub. No.: US 2026/0256676 A1
(43) Pub. Date: Sep. 3, 2026

(54) PEEL-OFF COSMETIC STAIN

(71) Applicant: Sacheu Beauty Inc., Studio City, CA (US)

(72) Inventors: Quinn Roukema, Studio City, CA (US); Sarah Cheung, Los Angeles, CA (US)

(21) Appl. No.: 19/654,174

(22) Filed: Apr. 21, 2026

Related U.S. Application Data
(63) Continuation-in-part of application No. 19/334,451, filed on Sep. 19, 2025.
Application No. 19/334,451 is a continuation of application No. 18/675,021, filed on May 27, 2024.
(60) Provisional application No. 63/469,388, filed on May 27, 2023.

(51) Int. Cl. A61K 8/81; A61K 8/34; A61K 8/37; A61K 8/49; A61K 8/73; A61Q 1/04; A61Q 19/00

(52) U.S. Cl. CPC A61K 8/8129; A61K 8/345; A61K 8/375; A61K 8/498; A61K 8/735; A61K 8/8111; A61Q 1/04; A61Q 19/001

(57) ABSTRACT

Lip liners and other cosmetic stains include a solvent (e.g., water) configured to evaporate substantially independently of a user’s skin, a film-forming agent (e.g., polyvinyl alcohol) dissolved in the solvent and configured to form a uniform film over the user’s skin as the solvent evaporates, and one or more colorants dissolved in the solvent and configured to stain the user’s skin. A color fixing agent (e.g., polyisobutene) enhances dispersion and penetration of the one or more colorants into the user’s skin. Other components may include glycerin (humectant, moisturizer, and emollient), diisostearyl malate (skin conditioning agent, lubricant, moisturizer, and emollient), tocopherol (including derivatives such as tocopheryl acetate) (antioxidant and emollient), hyaluronic acid (moisturizer and emollient), and phenoxyethanol (preservative).

### CROSS-REFERENCE TO RELATED APPLICATIONS

This application is a continuation-in-part of U.S. Patent Application No. 19/334,451, filed Sep. 19, 2025, which is a continuation of U.S. Patent Application No. 18/675,021, filed May 27, 2024, which claims the benefit of U.S. Provisional Application No. 63/469,388, filed on May 27, 2023, which are incorporated by reference in their entirety.

### BACKGROUND

Lip liner is a cosmetic used to touch up the outer edges of a user’s lips, often before applying lipstick or similar cosmetics, so that the user’s lips will appear more even in shape, more defined, and also to help prevent smearing of lipstick beyond the borders of the user’s lips.

Existing lip liners are made of waxes, oils, and colorants, and customarily are shaped as pencils in order to assist in drawing precise lines.

These existing products have many drawbacks. They wear off inconsistently over time, leading to the user’s lips appearing splotchy. Furthermore, these products can run or come off with use. This wear can also lead to inconsistent coloring on a user’s lips, along with smearing, i.e., lip liner being smeared onto the user’s face or fingers, as well as on other objects such as eating utensils or drinking glasses.

Many cosmetic products also use denatured alcohol (i.e., ethanol mixed with toxic or bitter additives to make it undrinkable), also known as “alcohol denat”, as an antimicrobial and emulsifying agent for other ingredients. However, alcohol denat is not suitable for long-term use on skin. Alcohol denat may evaporate quickly, and as a result may draw out moisture from the user’s skin, leaving the skin overly dry and causing irritation and damage to the skin’s barrier. Furthermore, long-term use of products containing alcohol denat can dissolve the skin’s surface sebum film, damaging the skin’s protective barrier, reducing skin tolerance, and causing sensitivity and irritation. Alcohol denat is particularly undesirable for a user’s lips, which have especially sensitive skin and so are more vulnerable to the damage alcohol denat may cause.

Example lip liners that are formulated with ethyl alcohol (ethanol) to accelerate drying are disclosed in Bae (EP 3159043 A1) and Jeong et al. (KR 20160087938 A). Bae requires 5 to 30 parts by weight of ethyl alcohol to provide desired drying characteristics, teaching that including less than 5 parts by weight of ethyl alcohol makes the volatilization rate so low as to significantly reduce the desired drying rate of the composition. Jeong et al. require 5.00 to 20.00 wt% of ethanol and teaches that if the concentration of ethanol is less than 5.00 wt%, the volatilization rate is slow, film drying time increases, and film forming takes a long time.

Other cosmetic stains are formulated to cover a user’s lips. For example, Kang et al. (KR20160101314A ) disclose a peel-off cosmetic composition for application to a user’s lips. This composition includes water, a film-forming agent, natural pigments, and an acrylic cross-linked polymer to enhance dispersion stability of the natural pigments, which are included as powders. A drawback of Kang et al. is that the natural pigments must be stabilized and maintained in dispersion by an acrylic cross-linked polymer, which forms a gel and lattice structure to trap the natural pigment within the lattice so that the natural pigment can be stably maintained a dispersed state. Rather than staining the lips, Kang teaches that the natural pigment is merely deposited on the surface of the lips. A substantial portion of the natural pigment, being embedded within the acrylic cross-linked polymer, is not transferred to the lips but remains in the peeled-off composition. This results in only slight coloration of the lips after peeling off the dried composition, as shown in FIGS. 1a and 1b. Moreover, the natural pigments deposited on the lips may be prone to smudging or rubbing off onto other objects. Kang also teaches that peeling off the film removes part of the stratum corneum of the lips.

There is accordingly a need for lip liner and other cosmetic stains that address some or all of the above issues.

### SUMMARY OF THE INVENTION

Embodiments of the present general inventive concept provide a liquid lip liner stain configured to stain a user’s lips a desired color, and a method of making the same. In some implementations, the lip liner stain is used to form a lip liner on at least a portion of the outer edges of a user’s lips. In other implementations, the lip liner stain can be used to cover a greater portion of the user’s lips, such as an entirety of the lips, similar to lipstick, to provide long-lasting color that does not smudge or rub off onto other objects, as typically occurs with conventional lipsticks.

In some embodiments, the film is configured such that, upon removal, it removes unabsorbed or non-penetrated colorant from the surface of the skin while leaving colorant embedded within the skin, resulting in a uniform stain with defined boundaries. Unlike systems in which pigments are suspended or trapped within a film matrix and remain on the surface of the skin, the present composition includes colorants dissolved within the solvent system that migrate into the skin during drying, such that removal of the film leaves a visible, transfer-resistant stain with defined boundaries.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a lip liner cosmetic stain including a solvent configured to evaporate substantially independently of a user’s skin, a film-forming agent dissolved in the solvent and configured to form a uniform or continuous film over the user’s skin as the solvent evaporates, and one or more colorants dissolved in the solvent and configured to stain the user’s skin a desired color.

In some embodiments, the solvent comprises or consists of water. The solvent may also include one or more cosmetically acceptable and water-miscible co-solvents, such as propylene glycol, dipropylene glycol, butylene glycol, pentylene glycol, or ethoxydiglycol (2-(2-ethoxyethoxy)ethanol). In preferred embodiments, the lip liner stain is substantially free of ethanol (e.g., contains less than about 1%). In more preferred embodiments, ethanol is omitted entirely from the lip liner stain.

In an embodiment, the solvent (i.e., water and optional co-solvent) may have a concentration between about 25 percent and about 55 percent by weight, or between about 30 percent and about 50 percent by weight, or between about 35 percent and about 45 percent by weight, or between about 37.5 percent and about 42.5% percent by weight, such as at a concentration of about 40.4 percent by weight.

In an embodiment, the film-forming agent may be configured to form a plurality of filaments as the solvent evaporates, the plurality of filaments being configured to form the film over the user’s skin. The film may be configured to be peeled off of the user’s skin after the solvent has evaporated.

In an embodiment, the film-forming agent may have a concentration by weight between about 5 percent and about 20 percent. In an embodiment, the film-forming agent may include or consist of polyvinyl alcohol. The polyvinyl alcohol may have a concentration between about 5 percent and about 20 percent by weight.

In an embodiment, the solvent may be configured to evaporate from the user’s skin between about 10 minutes and about 15 minutes after application thereto. In other words, enough of the solvent may evaporate from the user’s skin between about 10 minutes and about 15 minutes after application thereto to form a peelable film that can be peeled from the user’s lips.

In an embodiment, the lip liner stain may further include a color fixing agent configured to enhance the dispersion and penetration of the one or more colorants into the user’s skin. In some embodiments, the color fixing agent can have a concentration by weight of about 15 percent. In an embodiment, the color-fixing agent may include polyisobutene, which can be incorporated as part of an emulsion with the solvent (comprising or consisting of water) as a disperse phase. The polyisobutene may be used to ensure that the lip liner stain can evenly and completely cover the surface of the user’s skin during application, avoiding uneven application or missed spots. Polyisobutene may also help maintain the cohesiveness of the lip liner stain, and furthermore may ensure that the lip liner stain does not shatter during application and drying. The polyisobutene may have a concentration of about 15 percent by weight.

In an embodiment, the lip liner stain may further include a humectant dissolved in the solvent, the humectant being configured to absorb water from the environment, and a moisturizer dissolved in the solvent, the moisturizer being configured to form a moisture barrier over the user’s skin to contain water. An example humectant is glycerin. Other example humectants include other propylene glycol, butylene glycol, hexylene glycol, sorbitol, xylitol, maltitol, urea, glyceryl triacetate, and the like.

In an embodiment, the lip liner stain may further include a skin conditioning agent. In an embodiment, the skin conditioning agent may include diisostearyl malate, which can be incorporated as part of an emulsion with the solvent (comprising or consisting of water) as a disperse phase. The diisostearyl malate can be included at a concentration of about 5.6 percent by weight. It has been found that diisostearyl malate can also function as a color fixing agent, particularly in combination with polyisobutene.

In an embodiment, the lip liner stain may further include an antioxidant. In an embodiment, the antioxidant may include tocopherol (including derivatives such as tocopheryl acetate), which can be incorporated as part of an emulsion with the solvent (comprising or consisting of water) as a disperse phase, The skin conditioning can be included at a concentration of about 4.8 percent by weight.

In an embodiment, the lip liner stain may further include a preservative dissolved in the solvent at a concentration of about 0.1 percent by weight. An example preservative is phenoxyethanol.

The foregoing and/or other features and utilities of the present general inventive concept may be achieved by providing a method of manufacturing a lip liner stain, the method including providing a solvent at a concentration of about 40.4 percent by weight, dissolving a film-forming agent in the solvent at a concentration between about 5 percent and about 20 percent by weight, and dissolving one or more water-soluble colorants in the solvent at a concentration between about 0.2 percent and about 1.1 percent by weight.

In an embodiment, the method may further include comparing the combined solvent, film-forming agent, and one or more colorants with a color sample.

In an embodiment, the method may further include vacuum defoaming and filtering the combined film-forming agent, solvent, and one or more colorants to form a defoamed and filtered lip liner stain, which can be loaded in liquid form into storage or applicator containers.

In an embodiment, the method may further include dissolving a humectant into the solvent, the humectant being configured to absorb water from the environment, and dissolving a moisturizer into the solvent, the moisturizer being configured to form a moisture barrier over the user’s skin to contain water.

### BRIEF DESCRIPTION OF THE DRAWINGS

These and/or other features and utilities of the present general inventive concept will become apparent and more readily appreciated from the following description of the embodiments, taken in conjunction with the accompanying drawings of which:

FIG. 1 illustrates a lip liner application according to an exemplary embodiment of the present general inventive concept;

FIG. 2 illustrates a process of manufacturing a lip liner stain according to an exemplary embodiment of the present general inventive concept; and

FIGS. 3A-3C illustrate a process of applying and subsequently peeling off a lip liner stain according to an exemplary embodiment of the present general inventive concept.

### DETAILED DESCRIPTION OF EMBODIMENTS OF THE INVENTIVE CONCEPT

Reference will now be made in detail to embodiments of the present general inventive concept, examples of which are illustrated in the accompanying drawings, wherein like reference numerals refer to the like elements throughout. The embodiments are described below in order to explain the present general inventive concept while referring to the figures. Also, while describing the present general inventive concept, detailed descriptions about related well-known functions or configurations that may diminish the clarity of the points of the present general inventive concept are omitted.

Expressions such as “at least one of,” when preceding a list of elements, modify the entire list of elements and do not modify the individual elements of the list.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the detailed description of the invention. Thus, the terms used herein have to be defined based on the meaning of the terms together with the description throughout the specification.

Also, when a part "includes" or "comprises" an element, unless there is a particular description contrary thereto, the part can further include other elements, not excluding the other elements.

Hereinafter, one or more exemplary embodiments of the present general inventive concept will be described in detail with reference to accompanying drawings.

A lip liner stain 100 according to embodiments of the present general inventive concept may act as a stain, ingraining a desired color into a user’s skin, for example the skin of the user’s lips. The lip liner stain 100 may be applied to the user’s skin as a liquid, and then be allowed to dry, in the process forming a film 110 on the user’s skin. Once dry, the film 110 may be removed, such by peeling, leaving the user’s skin stained the desired color. Because the stain is ingrained into the user’s skin instead of being layered on over the skin, the color may last until it fades naturally, without smudging or coming off on other objects. In some embodiments, the film is configured such that, upon removal, it removes unabsorbed or non-penetrated colorant from the surface of the skin while leaving colorant embedded within the skin, resulting in a uniform stain with defined boundaries. Unlike systems in which pigments are suspended or trapped within a film matrix and remain on the surface of the skin, the present composition includes colorants dissolved within the solvent system that migrate into the skin during drying, such that removal of the film leaves a visible, transfer-resistant stain with defined boundaries.

A lip liner stain 100 according to embodiments of the present general inventive concept may be specially formulated to be safe to use around the sensitive skin of the lip area. To this end, a lip liner stain 100 according to embodiments of the present general inventive concept may lack alcohol denat (i.e., ethanol) or any similar components that can dry out and irritate skin, particularly the user’s lips. Embodiments may instead include components which avoid drying out or damaging the user’s skin, and may instead nourish, moisturize, and heal the user’s skin. For this reason, the lip liner stain 100 may be re-applied as desired to ensure the color on the user’s skin remains consistent, without damaging or drying out the user’s skin.

A lip liner stain 100 according to embodiments of the present general inventive concept may include a solvent such as water at a concentration between about 25 percent and about 55 percent, or between about 30 percent and about 50 percent by weight, or between about 35 percent and about 45 percent by weight, or between about 37.5 percent and about 42.5% percent by weight, such as a concentration of about 40.4% by weight. The solvent may also include one or more cosmetically acceptable and water-miscible co-solvents, such as propylene glycol, dipropylene glycol, butylene glycol, pentylene glycol, or ethoxydiglycol (2-(2-ethoxyethoxy)ethanol).

According to a preferred embodiment of the present general inventive concept, the solvent (primarily water) may be capable of evaporating in air at a standard air pressure of one atmosphere. The solvent may further be capable of evaporating substantially independently of the user’s skin, meaning that as the solvent evaporates it does not damage the user’s skin, and draws little to no moisture out of the skin. The solvent may assist in blending and dispersing other ingredients of the lip liner stain 100, serving as both a diluent and solvent within the lip liner stain 100. If ethanol is included, it is preferably included in an amount of less than 3 percent by weight, or less than 2 percent by weight, or less than 1 percent by weight, or it may be omitted entirely.

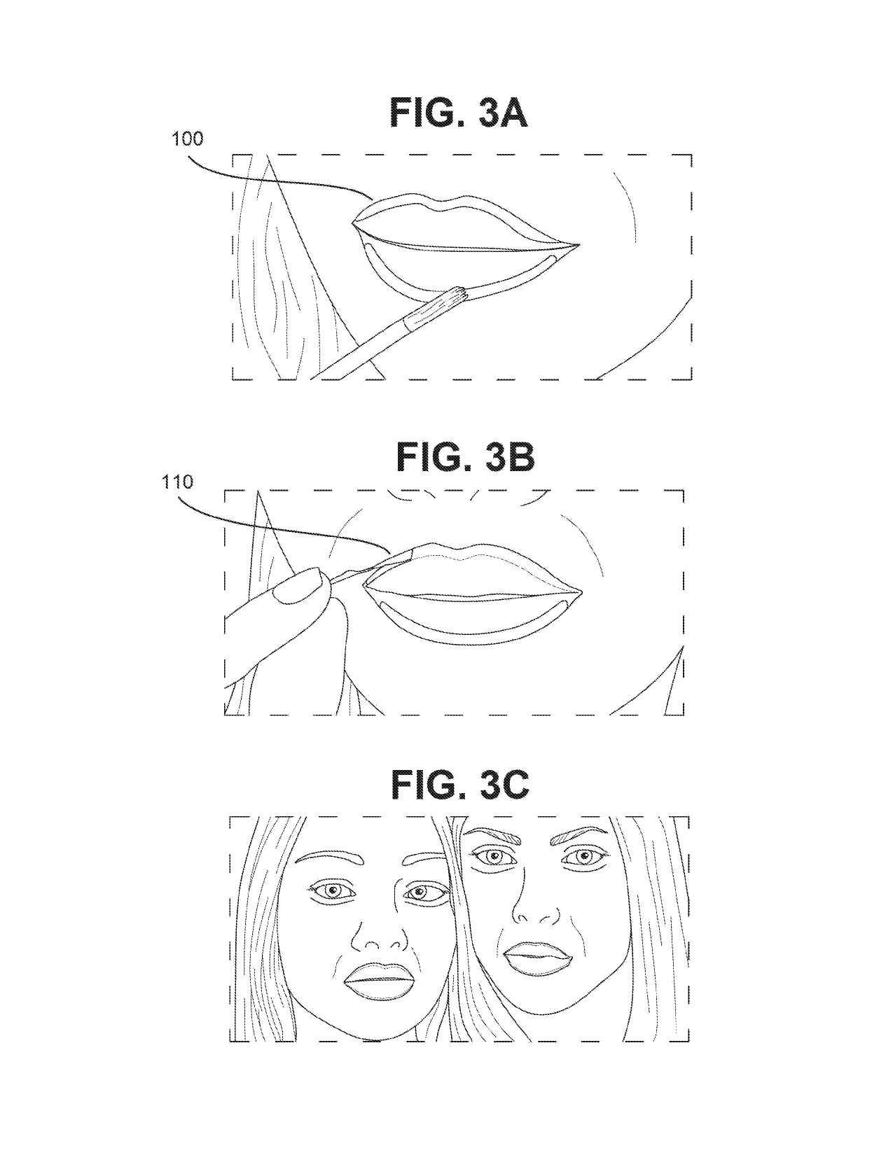

The lip liner stain 100 may further include a film-forming agent such as polyvinyl alcohol dissolved in the solvent at a concentration between about 5% and about 20% by weight, or between about 10% and about 20% by weight, or between about 12.5% and about 17.5% by weight. The film-forming agent may be selected from a group of materials capable of forming a physical film 110 over the surface of the lip liner stain 100 as the solvent evaporates in the air, as illustrated for example in FIG. 3B. The film-forming agent may form this film 110 without damaging or drawing moisture out of the user’s skin. Once dried, the film 110 may be peeled away, according to a process described in greater detail below. Polyvinyl alcohol is a chemically synthesized polymer which may be used as the film-forming agent in a preferred embodiment of the present general inventive concept. After being applied to a user’s skin, it may form filaments which come together to rapidly form a uniform, continuous, durable film 110 over the user’s skin surface as the moisture in the lip liner stain 100, including moisture from the solvent, evaporates.

A preferred embodiment of the lip liner stain 100 according to the present general inventive concept may further include one or more colorants, the colorant(s) being selected from a group of materials capable of penetrating the user’s skin and staining it a desired color after the lip liner stain 100 is applied thereto, while the solvent evaporates and the film-forming agent forms the film 110. According to embodiments of the present general inventive concept, the colorant(s) may be water soluble, safe for human skin, nontoxic, and non-bleeding, i.e., they have minimal “bleed” or flow beyond where they were applied to the user’s skin, in particular the user’s lips. Concentration of the colorant(s) may vary depending on the desired color in a given embodiment of the present general inventive concept. According to embodiments of the present general inventive concept, the lip liner stain 100 may include one or more colorants, each at a concentration between about 0.1% by weight and 2% by weight, or between about 0.15% by weight and 1.5% by weight, such as between about 0.2% by weight and 1.1% by weight. The water-soluble colorants are selected and formulated to stain the skin rather than coat the surface. Because the colorants are dissolved within the solvent system rather than being encapsulated within a gel lattice, they are able to migrate into the superficial layers of the skin during the drying and film-forming phase.

A preferred embodiment of the lip liner stain 100 according to the present general inventive concept may further include a color-fixing agent such as polyisobutene at a concentration between about 5 percent and about 25 percent by weight, or between about 7.5 percent and about 22.5 percent by weight, or between about 10 percent and about 20 percent by weight, or between about 12.5 percent and about 17.5 percent by weight, such as at a concentration of about 15% by weight. For the purposes of this description, “color fixing agent” is a component which may enhance the dispersion and penetration of the colorant into the user’s skin. As discussed herein, diisostearyl malate can also function as a color-fixing agent, particularly in combination with polyisobutene. The color-fixing agent ensures that by the time the surface film has fully formed and is ready to be peeled away from the lips, a substantial portion of the colorants have already penetrated into and been fixed within the skin. As a result, removal of the film does not remove the deposited stain, only residual colorant that has not already penetrated into and been fixed within the skin while the film is drying.

Polyisobutene is a very stable, high-viscosity synthetic resin which may be used to provide adhesion. According to embodiments of the present general inventive concept, polyisobutene may be used to ensure that the lip liner stain 100 can evenly and completely cover the surface of the user’s skin during application, avoiding uneven application or missed spots. Polyisobutene may therefore help to maintain the cohesiveness of the lip liner stain 100, and furthermore may ensure that the lip liner stain 100 does not shatter during application and drying. Furthermore, according to embodiments of the present general inventive concept, polyisobutene may be preferable to other color-fixing agents or adhesives, because polyisobutene may be extremely friendly to the user’s skin, causing very low irritation and without significant removal of the stratum corneum by the film-forming agent when the film is peeled off the lips. Still further, polyisobutene may not affect the performance or functions of other ingredients in the lip liner stain 100, making the entire formulation of the lip liner stain 100 more stable and effective.

A preferred embodiment of the lip liner stain 100 according to the present general inventive concept may further include additional components to provide color longevity, as well as moisturizers and emollients, which may soften or smooth a user’s skin. Examples of these additional components include glycerin, hyaluronic acid, and diisostearyl malate. Glycerin may be included at a concentration between about 5% and about 10% by weight, and may serve as a moisturizer and also promote color longevity. Glycerin may function as a humectant, helping to attract and lock in moisture, thereby helping to maintain a moisture balance of the user’s skin. The high-quality hydrating property of glycerin may help moisturize the user’s skin, which may help the lip liner stain 100 apply more smoothly to the user’s skin. Furthermore, glycerin may have hygroscopic properties, meaning it may absorb moisture from the air even in a dry environment. As such, glycerin may bring extra moisture to the user’s skin, which may help to repair dry and cracked skin. Examples of other humectants include propylene glycol, butylene glycol, hexylene glycol, sorbitol, xylitol, maltitol, urea, glyceryl triacetate, and the like.

Hyaluronic acid may be a moisturizer included in the lip liner stain 100 at a concentration between about 0.1% and about 3% by weight, or between about 0.5% and about 2% by weight. Hyaluronic acid is a natural polysaccharide present within the human body, and has exceptional moisture absorption capabilities, being able to absorb up to 1000 times its weight in water, and so may deeply hydrate and moisturize the user’s skin, keeping the skin soft and eliminating dryness or cracking. The addition of hyaluronic acid may therefore enhance the moisturizing performance of the lip liner stain 100, allowing a user’s skin to get deep nourishment while obtaining color from the colorant in the lip liner stain 100.

According to embodiments of the present general inventive concept, an overall performance of the lip liner stain 100 may be adjusted according to the ratio of glycerin and hyaluronic acid, as shown in Example 3, below. Changing the concentration of an ingredient of the lip liner stain 100, such as hyaluronic acid for example, may change the effect and feel of the lip liner stain 100. At too low a concentration of hyaluronic acid, the lip liner stain 100 may not sufficiently moisturize the user’s skin, potentially leading to dryness, discomfort, and ineffective product performance such as less lasting wear time or uneven color distribution. At a concentration of hyaluronic acid by weight of about 3%, the lip liner stain 100 may sufficiently moisturize the user’s skin to avoid dryness or cracking, and may offer a desired consistency of the lip liner stain 100. Conversely at concentrations of hyaluronic acid over 3%, the lip liner stain 100 may cause adverse reactions such as bumpy texture on the user’s skin, or can lead to product instability resulting in overflow, where the lip liner stain 100 does not adhere to the user’s skin and instead seeps beyond the area of application.

Diisostearyl malate may be included in the lip liner stain 100 at a concentration between about 3 percent and about 8 percent by weight, or between about 3.5 percent and about 7.5 percent by weight, or between about 4 percent and about 7 percent by weight by weight, or between about 4.5 percent and about 6.5 percent by weight, such as at a concentration of about 5.6% by weight. Diiosostearyl malate may be a skin conditioning agent and lubricant, and may moisturize and serve as an emollient, ensuring a smooth application and leaving a pleasant feel on the skin. Due to the emollient properties, diisostearyl malate may create a more stable and homogenous product by providing a better dispersion medium for the colorant(s). As a result, diisostearyl malate may promote color longevity, with color more evenly distributed on the user’s skin. Thus, diisostearyl malate can also function as a color-fixing agent.

A preferred embodiment of the lip liner stain 100 according to the present general inventive concept may also include tocopherol (including derivatives such as tocopheryl acetate) at a concentration of between about 2.5 percent and about 7 percent by weight, or between about 3 percent and about 6.5 percent by weight, or between about 3.5 percent and about 6 percent by weight, or between about 4 percent and about 5.5 percent by weight, such as about 4.8% by weight. Tocopherol, which may also be known as “Vitamin E,” possesses antioxidant capabilities, and may protect the user’s lips from the potential harm of pigments and environmental factors.

A preferred embodiment of the lip liner stain 100 according to the present general inventive concept may also include a preservative such as phenoxyethanol at a concentration between about 0.02 percent and about 0.5 percent by weight, or between about 0.04 percent and about 0.4 percent by weight, or between about 0.06 percent and about 0.3 percent by weight, or between about 0.08 percent and about 0.2 percent by weight, such as at a concentration of about 0.1% by weight. The preservative may ensure the lip liner stain 100 does not harbor bacteria or mold during its shelf life.

In a preferred embodiment of the present general inventive concept, the solvent may dissolve or disperse all the components of the lip liner stain 100 to form a homogenous mixture or emulsion. Components that are insoluble in water, such as polyisobutene, diisostearyl malate, and tocopherol (or derivative thereof) can be incorporated as part of an emulsion or dispersed phase within the system to ensure uniform distribution despite being insoluble in water. In combination, the ingredients of the lip liner stain 100 may help the lip liner stain 100 provide an even protective layer on the user’s skin, while also ensuring a natural coloration. The combination of ingredients therefore may provide both a desirable makeup effect and nourishment to the user’s skin.

According to exemplary embodiments of the present general inventive concept, the homogenous mixture or emulsion of the lip liner stain 100 may have a liquid consistency rather than a gel. A user may apply this liquid lip liner stain 100 to a desired portion of their skin with a precision applicator, e.g., a small brush, as illustrated for example in FIG. 3A.

Once exposed to air, the solvent (i.e., water) in the lip liner stain 100 may begin to evaporate, causing the lip liner stain 100 to dry on the user’s skin. During this drying time, colorants in the lip liner stain 100 may penetrate into and stain the user’s skin, thereby imparting the desired color to the user’s skin. Without alcohol denat (ethanol), which encourages evaporation, a lip liner stain 100 according to embodiments of the present general inventive concept may take a relatively longer time to dry than a conventional lip liner, for example 10-15 minutes. This extended drying time may give relatively more time for the one or more colorants in the lip liner stain 100 to be imparted to the user’s skin, resulting in a more consistent color, as well as a color that lasts longer by virtue of deeper penetration into and staining of the user’s skin. While the lip liner stain 100 is drying, the film-forming agent may form a thin film 110 over the surface of the lip liner stain 100. This film 110 may adhere to the user’s skin as the solvent evaporates fully, thereby ensuring that the lip liner stain 100 remains in place and only the skin the user originally applied the lip liner stain 100 to is stained. Once the lip liner stain 100 has been allowed to dry on the user’s skin for a desired time to impart color, the film 110 may be peeled off of the user’s skin, as illustrated for example in FIG. 3B. When the film 110 is peeled off, it exposes the user’s skin, as well as the developed color on the skin, as illustrated for example in FIG. 3C. Removal of the film 110 may also remove any excess lip liner stain 100 which had not yet dried, including removing colorant that has not penetrated into the skin.

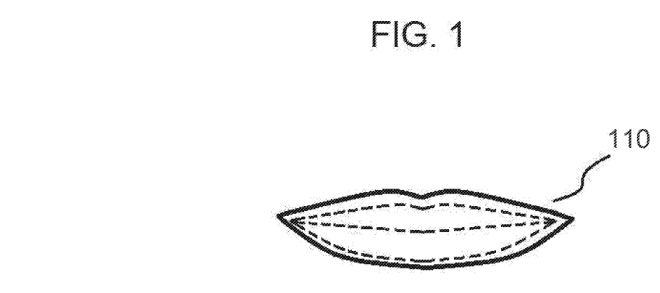

As illustrated in FIG. 1, the film 110 of lip liner stain 100 may help define a border around a desired area, for example a user’s lips, which are illustrated in FIG. 1 as dashed lines. While the film 110 is in place, a user may apply cosmetic products, e.g., lipstick, to the desired area, and the film 110 may act as a border or barrier to prevent “bleed,” i.e., the cosmetic products extending beyond the area defined by the lip liner stain 100. According to an exemplary embodiment of the present general inventive concept, the film 110 formed by the lip liner stain 100 may catch any “bleed.” For example, lipstick extending past the user’s lips may fall on the film 110 of the lip liner stain 100, and this bleed may be removed with the film 110. As such, a user may use the lip liner stain 100 to define an exact area where they want to apply cosmetic products, and may remove any bleed or errors in applying any such cosmetic products to the defined area. Removing the film may also remove excess colorant that has not penetrated into and stained the skin, ensuring more even straining of the lips.

It will be understood that the applications illustrated in FIG. 1 and FIGS. 3A-3C are only exemplary embodiments of the present general inventive concept. According to other exemplary embodiments, the lip liner stain 100 may be applied over a larger area, for example the entire surface of the user’s lips, similar to lipstick, such that the lip liner stain 100 stains the user’s skin the desired color to provide long-lasting color that does not smudge or rub off onto other objects, as typically occurs with conventional lipsticks. According to this exemplary embodiment, the lip liner stain 100 may be applied in lieu of other cosmetics, rather than in addition to them.

In conventional cosmetics, fixing agents are commonly used in products such as setting sprays and powders to enhance wear time or accelerate drying. These agents often include drying components, such as talc or alcohol. In contrast, the disclosed lip liner stains incorporate polyisobutene and diisostearyl malate, which are non-drying and function as emollient fixing agents rather than traditional drying fixatives. These emollients enhance the dispersion and controlled penetration of the colorants into the skin, resulting in longer-lasting color and a more uniform finish on the lips. Additionally, these emollients help protect the stratum corneum during the peel-off stage. This allows the surface film to be removed while leaving the deposited color intact, without stripping or damaging the underlying lip tissue (i.e., peeling the surface film does not remove the stratum corneum).

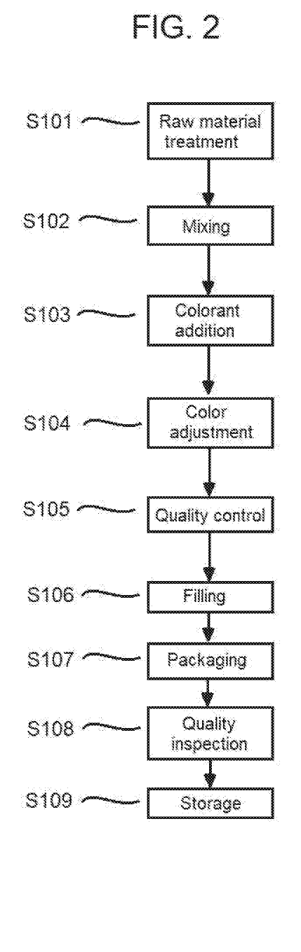

FIG. 2 illustrates a process of manufacturing a lip liner stain 100 according to an exemplary embodiment of the present general inventive concept. As illustrated therein, according to exemplary embodiments of the present general inventive concept, at step S101 of the process, raw materials, including the film-forming agent and solvent, may be treated with ultraviolet radiation to sterilize them and prepare them for processing.

At step S102 of the process, the treated raw materials may be added to a thermostatic stirring pot according to the formula, and may be stirred and melted uniformly at a constant temperature. According to exemplary embodiments of the present general inventive concept, the stirring pot may be made of stainless steel or other non-reactive material suitable for high temperatures.

Following step S102 of the process, the material may be substantially transparent and uniform, i.e., the various components may be thoroughly mixed together to be homogenous. Once the material has reached this state, in step S103 of the process one or more colorants may be weighed, melted, and added to the stirring pot according to formula requirements.

At step S104 of the process, the material with colorant added may be compared with an original sample to double-check the color. If a color difference is detected, additional colorants may be added to the material until the resulting material matches the original sample. At this point the colored material may be vacuum defoamed and filtered. This step may also be called “percolation.”

At step S105, the material may be inspected for quality, which may include inspecting the material for color, viscosity, total number of microorganisms, etc. After it is confirmed that the material meets preset quality requirements, the material may be stored and sterilized via ultraviolet radiation.

At step S106, the material may be filled and produced according to a desired order. According to an exemplary embodiment of the present general inventive concept, this filling and producing may comprise loading the processed material into one or more storage containers, for example a tube.

At step S107, the filled products may be packaged, i.e., the storage containers may be put into retail packaging for sale. At step S108 the packaged products may undergo a quality control inspection to detect any quality issues. Finally at step S109 the inspected packaged products may be put into storage, where they may await transport and delivery to a final consumer.

A lip liner stain according to embodiments of the present general inventive concept may allow users to impart a desired color to their skin without concerns of the color coming off or smearing with use, and also without risk of damaging or drying out their skin. Embodiments of the present general inventive concept may also allow a user to more precisely apply cosmetic products, avoiding bleed.

### Example 1

A lip liner stain was prepared with water as a solvent at a concentration of 40.4% by weight and polyvinyl alcohol as a film-forming agent at a concentration of 20% by weight. Polyisobutene as a color-fixing agent was added at a concentration of 15% by weight. Diisostearyl malate was added at a concentration of 5.6% by weight. Tocopherol was added at a concentration of 4.8% by weight. Phenoxyethanol was added at a concentration of 0.1% by weight. The colorants used were CI 16035 at a concentration of about 0.4% by weight, CI 45410 at a concentration of about 0.3% by weight, CI 19140 at a concentration of about 0.2% by weight, and CI 42090 at a concentration of about 0.2% by weight.

To prepare the lip liner stain, the ingredients were mixed in groups. The first ingredients added to the stirring pot were “Group A” ingredients, including the solvent and color-fixing agent, as well as diisostearyl malate and tocopherol. These Group A ingredients were added to the stirring pot, heated to a temperature of about 50 degrees Celsius, and stirred at a rate of about 100 rpm. The Group A ingredients were mixed under these conditions for about 20 minutes.

After the Group A ingredients were mixed, “Group B” ingredients were added to the stirring pot. “Group B” ingredients include the film-forming agent. Once the Group B ingredients were added, the stirring pot was heated to about 95 degrees Celsius and stirred at a rate of about 100 rpm. The combination of Group A and Group B was stirred under these conditions for about 30 minutes. The process of mixing and combining ingredient Groups A and B would take place during step S102, or “Mixing,” of the process illustrated in FIG. 2.

Once Group A and Group B ingredients were mixed together, they were mixed with “Group C ingredients,” which include the colorants and phenoxyethanol. The mixed Group A, B, and C ingredients were put into a sieving grinding machine and ground 1-3 times, depending on whether the colorants, which were powders, had been ground evenly. The process of adding Group C ingredients and grinding them would take place during step S103, or “Colorant Addition,” of the process illustrated in FIG. 2.

Following the grinding, the lip liner stain was completed according to the process illustrated in FIG. 2, after which it was ready for application. Once the lip liner stain was applied to the user’s skin and allowed to dry, a process taking approximately 15 minutes, the film was peeled off of the user’s skin. The film was solid enough to be removed as one piece. The lip liner successfully stained the user’s skin the desired color.

### Example 2

A lip liner stain was prepared with identical formulation and process to Example 1, except it included polyvinyl alcohol at a concentration of 5% by weight. The lip liner stain was applied to the user’s skin and allowed to dry. Drying time was similar to that of Example 1. The film was easier to remove from the user’s skin than in Example 1, but the film was more fragile and did not come off smoothly in one piece. The lip liner stain still successfully stained the user’s skin the desired color.

### Example 3

A lip liner stain was prepared with an identical formulation as Example 1, further including glycerin at a concentration of 10% by weight, and hyaluronic acid at a concentration of 3% by weight. The process for manufacture was identical to Example 1, except that hyaluronic acid was included in “Group A” ingredients, while glycerin was included in “Group B” ingredients.

It was found that the lip liner stain with this formulation provided both durable color and ample moisture retention, providing the user a comfortable feeling. There was minimal overflow or adverse reactions in the user’s skin. Glycerin and hyaluronic acid were found to assist in each others’ functions at these concentrations. Glycerin as a humectant pulled in water molecules from the environment, and hyaluronic acid acted as a “moisture barrier,” locking the water molecules in to prevent evaporation.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

What is claimed is:

1. A liquid peel-off lip liner stain, comprising: a solvent configured to evaporate substantially independently of a user’s skin after the lip liner stain is applied to a user’s lips, wherein the solvent comprises water and is substantially free of ethanol or contains less than about 1% ethanol by weight of the lip liner stain;a film-forming agent dissolved in the solvent and configured to form a continuous film over the user’s skin as the solvent evaporates, the film being configured to be peeled from the user’s lips after drying; one or more colorants dissolved in the solvent and configured to penetrate into the user’s skin during evaporation of the solvent; and a color fixing agent configured to enhance dispersion and penetration of the one or more colorants into the user’s skin, wherein the lip liner stain is initially a liquid for application to the user’s lips, forms the film upon drying, and upon removal of the film removes unabsorbed colorant from the surface of the lips while leaving colorant embedded within the skin to provide a stain.

2. The liquid peel-off lip liner stain of claim 1, wherein the film-forming agent is configured to form the film during evaporation of the solvent such that the one or more colorants penetrate into the user’s skin prior to complete formation of the film.

3. The liquid peel-off lip liner stain of claim 1, wherein removal of the film removes excess composition from the surface of the user’s lips while leaving the colorant embedded within the skin substantially intact.

4. The liquid peel-off lip liner stain of claim 1, wherein the stain remaining after removal of the film exhibits defined boundaries corresponding to an area of application of the lip liner stain.

5. The liquid peel-off lip liner stain of claim 1, wherein the colorant embedded within the skin is resistant to transfer to external surfaces after removal of the film.

6. The liquid peel-off lip liner stain of claim 1, wherein the lip liner stain contains less than about 1% ethanol by weight.

7. The liquid peel-off lip liner stain of claim 1, wherein the lip liner stain comprises an emulsion or dispersion in which water-insoluble components are distributed within the solvent.

8. The liquid peel-off lip liner stain of claim 1, wherein the film-forming agent comprises polyvinyl alcohol, wherein the polyvinyl alcohol forms a plurality of filaments as the solvent evaporates, the plurality of filaments forming the film over the user’s skin.

9. The liquid peel-off lip liner stain of claim 8, wherein the polyvinyl alcohol has a concentration between about 5 percent and about 20 percent by weight, or between about 10 percent and about 20 percent by weight, between about 12.5 percent and about 17.5 percent by weight.

10. The liquid peel-off lip liner stain of claim 1, wherein the water has a concentration between about 25 percent and about 55 percent by weight, or between about 30 percent and about 50 percent by weight, or between about 35 percent and about 45 percent by weight, or between about 37.5 percent and about 42.5% percent by weight.

11. The liquid peel-off lip liner stain of claim 1, wherein the one or more water-soluble colorants have a concentration between about 0.1% by weight and 2% by weight, or between about 0.15% by weight and 1.5% by weight, or between about 0.2% by weight and 1.1% by weight.

12. The liquid peel-off lip liner stain of claim 1, the color fixing agent comprises polyisobutene.

13. The liquid peel-off lip liner stain of claim 12, wherein the polyisobutene has a concentration between about 5 percent and about 25 percent by weight, or between about 7.5 percent and about 22.5 percent by weight, or between about 10 percent and about 20 percent by weight, or between about 12.5 percent and about 17.5 percent by weight, or about 15% by weight.

14. The liquid peel-off lip liner stain of claim 1, further comprising glycerin at a concentration between about 5% and about 10% by weight.

15. The liquid peel-off lip liner stain of claim 1, further comprising diisostearyl malate at a concentration between about 3 percent and about 8 percent by weight, or between about 3.5 percent and about 7.5 percent by weight, or between about 4 percent and about 7 percent by weight by weight, or between about 4.5 percent and about 6.5 percent by weight, or about 5.6% by weight.

16. The liquid peel-off lip liner stain of claim 1, further comprising tocopherol or tocopheryl acetate at a concentration of between about 2.5 percent and about 7 percent by weight, or between about 3 percent and about 6.5 percent by weight, or between about 3.5 percent and about 6 percent by weight, or between about 4 percent and about 5.5 percent by weight, or about 4.8% by weight.

17. The liquid peel-off lip liner stain of claim 1, further comprising hyaluronic acid at a concentration between about 0.1% and about 3% by weight, or between about 0.5% and about 2% by weight.

18. The liquid peel-off lip liner stain of claim 1, further comprising phenoxyethanol at a concentration between about 0.02 percent and about 0.5 percent by weight, or between about 0.04 percent and about 0.4 percent by weight, or between about 0.06 percent and about 0.3 percent by weight, or between about 0.08 percent and about 0.2 percent by weight, or at a concentration of about 0.1% by weight.

19. A liquid peel-off lip liner stain, comprising: a solvent configured to evaporate substantially independently of a user’s skin after the lip liner stain is applied to a user’s lips, wherein the solvent comprises water and omits ethanol; a film-forming agent dissolved in the solvent and configured to form a continuous film over the user’s skin as the solvent evaporates, the film-forming agent comprising polyvinyl alcohol; one or more colorants dissolved in the solvent and configured to penetrate into the user’s skin during evaporation of the solvent; polyisobutene; and a plurality of components selected from glycerin, diisostearyl malate, tocopherol, hyaluronic acid, and phenoxyethanol, wherein the lip liner stain is initially a liquid for application to the user’s lips, forms a film that can be peeled off the user’s lips after the solvent has evaporated, and upon removal of the film removes unabsorbed colorant from the surface of the lips while leaving colorant embedded within the skin to provide a stain.

20. The liquid peel-off lip liner stain of claim 19, wherein the water has a concentration between about 30 percent and about 50 percent by weight, and wherein the solvent comprises one or more cosmetically acceptable and water-miscible co-solvents selected from propylene glycol, dipropylene glycol, butylene glycol, pentylene glycol, and ethoxydiglycol..

21. The liquid peel-off lip liner stain of claim 19, wherein the polyvinyl alcohol has a concentration between about 5 percent and about 20 percent by weight.

22. The liquid peel-off lip liner stain of claim 19, wherein the polyisobutene has a concentration between about 5 percent and about 25 percent by weight.

23. The liquid peel-off lip liner stain of claim 19, wherein the lip liner stain comprises glycerin at a concentration between about 5% and about 10% by weight.

24. The liquid peel-off lip liner stain of claim 19, wherein the lip liner stain comprises diisostearyl malate at a concentration between about 3 percent and about 8 percent by weight.

25. The liquid peel-off lip liner stain of claim 19, wherein the lip liner stain comprises tocopherol or tocopheryl acetate at a concentration of between about 2.5 percent and about 7 percent by weight.

26. A liquid peel-off lip liner stain, comprising: a solvent configured to evaporate substantially independently of a user’s skin after the lip liner stain is applied to a user’s lips, wherein the solvent comprises water at a concentration between about 30 percent and about 50 percent by weight; a film-forming agent dissolved in the solvent and configured to form a continuous film over the user’s skin as the solvent evaporates, the film-forming agent comprising polyvinyl alcohol at a concentration between about 5 percent and about 20 percent by weight; one or more colorants dissolved in the solvent at a concentration between about 0.1% by weight and 2% by weight and configured to penetrate into the user’s skin during evaporation of the solvent; polyisobutene at a concentration so as to enhance dispersion and penetration of the one or more colorants into the user’s skin;glycerin at a concentration so as to function as a humectant and skin moisturizer; diisostearyl malate at a concentration so as to function as a skin conditioning agent; tocopherol or tocopheryl acetate at a concentration so as to function as an antioxidant; hyaluronic acid at a concentration between about 0.1% and about 3% by weight so as to function as a skin moisturizer; and phenoxyethanol at a concentration so as to function as a preservative, wherein the lip liner stain is initially a liquid for application to the user’s lips, forms a film that can be peeled off the user’s lips after the solvent has evaporated, and upon removal of the film removes unabsorbed colorant from the surface of the lips while leaving colorant embedded within the skin to provide a stain.
